(19) United States
(12) Patent Application Publication
(10) Pub. No.: US 2012/0277622 A1
(43) Pub. Date: Nov. 1, 2012

(54) METHOD AND APPARATUS FOR VISUAL STIMULATION AND RECORDING OF THE PATTERN ELECTRORETINOGRAM OF THE VISUAL EVOKED POTENTIALS

(75) Inventor: Gualtiero REGINI, Roma (IT)

(73) Assignee: INGENESI OF GUALTIERO REGINI, Roma (IT)

(21) Appl. No.: 13/449,335

(22) Filed: Apr. 18, 2012

(30) Foreign Application Priority Data
Apr. 28, 2011 (IT) RM2011A000221

(51) Int. Cl. A61B 3/10

(52) U.S. Cl. 600/558

(57) ABSTRACT

A method and apparatus for visual stimulation and recording of the pattern electroretinogram (PERG) and of the visual evoked potentials (VEP), by way of transient and steady-state visual stimuli, including a simultaneous transient and steady-state stimulation of a plurality of zones of the visual field visualization element, and an algorithm able to reconstruct the second harmonic component of the steady-state signal and the transient signal of the zones.

### FIELD OF THE INVENTION

The present invention applies to the field of diagnostic instruments for testing visual functions and in particular for recording pattern electroretinogram (PERG) and visual evoked potentials (VEP).

### STATE OF THE ART

Since many years, the instruments for the diagnosis of diseases or for research in biological and cellular mechanisms of human and animal visual systems have made use of photopic stimulators and related apparatuses for recording VEP and PERG.

In order to perform these electrofunctional recordings, the subjects are placed in front of various systems for image visualisation, such as cathode ray tube (CRT) displays, liquid crystal displays (LCD), video projectors, LCD glasses, arrays of LED diodes, etc.

Appropriate electrodes are applied to the subjects, in positions specific for each type of recording.

Then, stimulation patterns are administered and changed as a function of time. The periodic variations in the contrast of the stimulation patterns induce variations in electric potentials in each examined subject at retinal and cortical levels. These electrophysiology techniques are well known and recognised as part of medical practice and scientific research.

In accordance with the guidelines of International Society for Clinical Electrophysiology of Vision (ISCEV) (ISCEV Standard for full-field clinical electroretinography (2008 update) Marmor M F, Fulton A B, Holder G E, Miyake Y, Brigell M, Bach M; International Society for Clinical Electrophysiology of Vision. Doc Ophthalmol. 2009 Febraury;118(1):69-77. Epub 2008 Nov. 22), the recording of PERG is defined as transient type when the temporal frequency of the structured stimulus is lower than 3 Hz, and it is defined as steady-state when the stimulus frequency is higher than 5 Hz. In the steady-state PERG, the useful information from responses is made by the sinusoidal component F2 with a frequency equal to twice the frequency of F1 stimulation. Transient and steady-state modes are used especially for the study of glaucoma.

In most of medical and scientific papers, the use of PERG for the early diagnosis of glaucoma is focused on either transient stimulation or steady-state stimulation, while in a few papers both modes have been combined in an attempt to increase sensitivity and specificity (e.g. “Pattern electroretinograms from hemifields in normal subjects and patients with glaucoma” Graham S L, Wong V A, Drance S M, Mikelberg F S., Investigative Ophthalmology & Visual Science, August 1994, Volume 35, No. 9, 3347-56; WO2006/106548 A1-Baglini).

For this purpose, the PERG response is examined in two or more areas of the visual field of the same subject. A main advantage introduced by said examination in two or more areas of the visual field consists of reducing the intersubjective variability in PERG response amplitude, based on the comparison between amplitudes of the PERG response in different areas of the visual field in the same subject. Another advantage, as already mentioned, is to consider both abnormal steady-state responses and abnormal transient responses in each subject.

However there is a drawback in recording electrofunctional responses from two or more areas of the visual field that consists of recording said responses in subsequent times. If e.g. the central viewing area called EE is divided in two hemifields, a lower one called Ei and an upper called Es, just one hemifield at a time is stimulated, equal to 50% of the entire area, while the other hemifield is maintained at a constant illumination; it follows that the duration of the examination of two hemifields Ei and Es is double, with respect to the recording of the whole central visual area EE. Furthermore, as known, the halving of the stimulated surface worsens the signal-to-noise ratio.

Further, the use of more complex techniques of visual stimulation, as described in patent application US2008/0108908—Madness et al., or as the PERG and VEP multifocal described in U.S. Pat. No. 4,846,567—Sutter, can not optimize the duration of the examination. The reason of this is that in said techniques the visual stimulus pattern is active, at each video frame, only on a part of the total examined area, typically about 50% of the total area in the case of multifocal PERG and VEP in U.S. Pat. No. 4,846,567 and about 25% of the total area in U.S.2008/0108908. Furthermore, said techniques provide only transient type recording and do not provide steady-state recording. Finally, there are methods that allow the stimulation and the PERG or VEP recording of the whole visual area, divided in a plurality of simultaneously stimulated zones, as described in the patent U.S. Pat. No. 5,539,482—James et al.—or as described in publications that refer to the technique of “cyclic summation” as “Pattern reversal ERG with LED-stimulation using cyclic summation technique. Link B, Jünemann A, Horn F K, Doc Ophthalmol. 2006 January;112(1):53-60. However, the drawback in all these methods is that the stimuli are only of a steady-state type, but not of a transient type. In fact, these methods are based on the separation of single spectral components of the different stimulation steady-state frequencies, each of which is uniquely related to the respective area of stimulation. In the state of the art the simultaneous transient and steady-state stimulation on different areas has not been reported, due to the problem that the spectral components of the recorded signal are not uniquely related to the relative areas of stimulation.

### SUMMARY OF THE INVENTION

The apparatus and the method of the present invention are used for electrofunctional PERG or VEP recording on different zones that are simultaneously stimulated in transient and in steady-state mode. In its preferred embodiment PERG from two hemifields that are the upper and lower hemifields of the retina of the examined subject, is described in order to obtain the transient and steady-state responses of said hemifields. The measurement of parameters like amplitudes, latencies, and amplitude ratios of transient and steady-state responses will be obtained by means of the apparatus according to the invention in normal subjects and in patients and then compared to evaluate its usefulness for the diagnosis of glaucoma.

A main advantage of the present invention with respect to conventional testing methods is to minimise the recording times of transient and steady-state PERG on two hemifields, since the stimuli are administered simultaneously, rather than in subsequent times.

The examination of the electrophysiological response of the visual field is thus reduced to only two sessions of stimulation, at the end of which there are four responses, two of transient type and two of steady-state type for two visual hemifields.

A further advantage of the present invention is to allow to examine a greater number of areas of the visual field, and then to have a more detailed mapping of functional vision, with an advantage consisting, also in this case, in a reduction of time with respect to traditional methods.

Furthermore, an advantage of the present invention is to collect simultaneously the responses from two distinct areas of the visual field without the drawback that the patient physiological response changes form one session to the next one due to well-known factors such as patient adaptation, change of focusing, muscle movement artifacts.

The present method is also characterised by a rule of synchronization of two different patterns presented simultaneously on the screen of a photopic stimulator, the first pattern of them eliciting the transient component and the second one eliciting the steady-state component of the stimuli. The present method is also characterised by an algorithm of reconstruction of the transient signal of the response and of the second harmonic component of the steady-state signal of the response. Said method and the apparatus that implements it, will be described in detail in the following.

### BRIEF DESCRIPTION OF THE DRAWINGS

FIG. 1 shows the recording apparatus in its preferred embodiment;

FIG. 2 shows waveforms of input and output signals of the apparatus;

FIG. 3 shows a block diagram of the program implemented in the apparatus; and

FIG. 4 shows a stimulation sequence for each quadrant.

### DESCRIPTION OF PREFERRED EMBODIMENT

For embodying the invention, it is necessary to use a means of visual stimulation capable of projecting onto the retina of a subject, two separate and different patterns, one of which is a transient PT pattern and the other one is a steady-state PS pattern, having an average luminance that is constant over time, according to the ISCEV guidelines for the stimulation PERG and VEP. Said patterns suitable for simultaneously stimulating two different zones of the retina of the subject consist for example of horizontal or vertical bars, checkerboard, triangles, hexagons or other geometrical elements, where light and dark elements invert their contrast periodically over time.

CRT displays or LCDs, video projectors, micro-display glasses, LED arrays are among the devices of visual stimulation most frequently used.

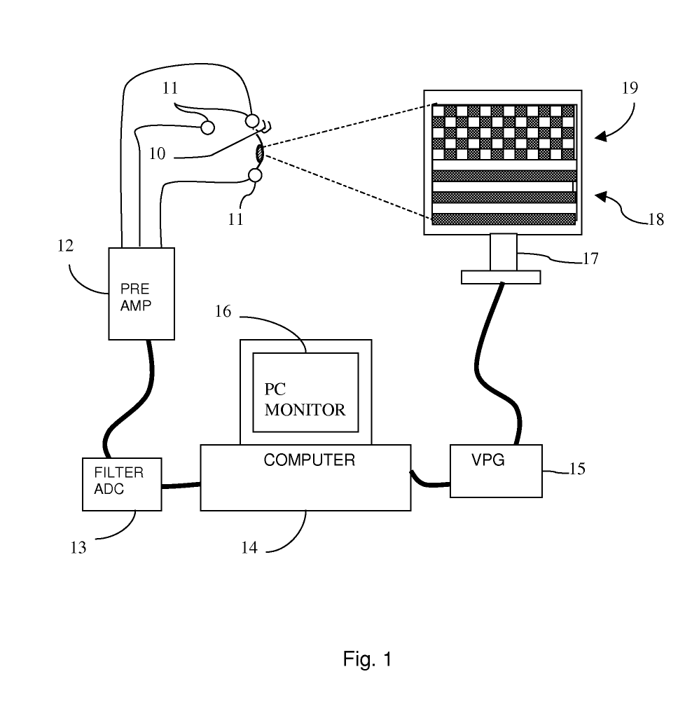

In the present preferred embodiment of the invention as represented by the apparatus in FIG. 1, there are: a stimulating display 17, whose surface is divided in two zones, where the upper half 19 is initially used for the PT pattern, and the lower half 18 for the PS pattern. The two patterns are produced by a video generator VPG 15, such as a common graphic card, controlled by a computer. By way of a non limiting example, the PS pattern is formed by horizontal bars with spatial frequency of 1.6 cycles/degree and has a Tss time period suitable for the stimulation of steady-state PERG. The PT pattern, formed by a checkerboard with spatial frequency equal to 1.0 cycle/degree, has a Ttr time period suitable for the stimulation of transient PERG.

Said patterns are subjected to contrast modulation or periodic contrast inversion, according to the respective periods Ttr and Tss.

A feature of the present invention is that said periods Ttr and Tss must comply with the following rule of synchronisation:

Ttr=Tss(2k+1)/2

being k an arbitrary integer; this rule allows a reconstruction algorithm of transient and steady-state signals to be effected properly as described below; this rule also makes possible to synchronise the frame rate of the stimulator with the frequency of steady-state as well as of the transient signal. In practice, the minimum period of steady-state stimulation signal is about 50 milliseconds, while the maximum period of the transient stimulation signal is 4000 ms equivalent to 0.25 Hz, so the useful range of the said integer constant k is included between 1 and about 80.

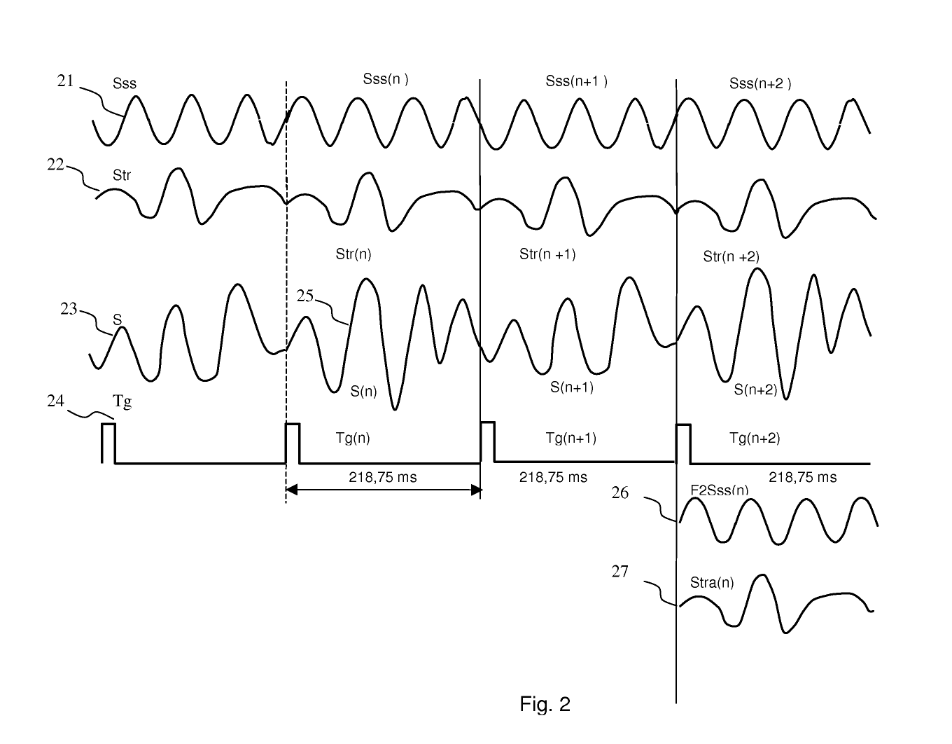

There are a plurality of electrodes 11 for signal acquisition, the electrodes being selected from those normally used for PERG recording, which are applied to the subject 10 which stares at a cross in the centre of the stimulation display 17; the signal received by said electrodes is amplified by a preamplifier 12, filtered by an ADC device 13 to limit its maximum frequency, in the specific example 30 Hz with a 2nd order filter, so as to reduce the noise of the electrical supply network, and then digitised by the 16-bit A/D converter present in said ADC device 13; the digitised signal is then sent to a computer 14 for data acquisition, processing and control, which has a display 16 for the operator and a program which, as illustrated in FIG. 2, allows reconstruction and visualisation of graphs and measures of the second harmonic component of the steady-state called F2Sss, and of the transient signal called Stra.

FIG. 2 shows the waveforms of the input and output signals of the system of acquisition. The trace 23 shows the signal S, picked up by the electrodes and amplified by the preamplifier during stimulation carried out by said patterns PT and PS. Said signal S results from the linear overlap of the virtual transient stimulation signal Str 22, due to the pattern PT, and of virtual steady-state stimulation signal Sss 21, due to the pattern PS.

Signals Str and Sss are defined as virtual because they are not physically present in the output of the preamplifier 12, where the sole S signal is present that is the sum of said signals Str and Sss.

In particular, virtual signal Sss 21, e.g. depicted in FIG. 2, has period Tss=125 ms corresponding to a PS pattern stimulation frequency of 8 Hz that is suitable for steady-state stimulation and it is mainly made of the second harmonic sinusoidal component, having a period equal to 62.5 ms. The virtual transient stimulation signal Str 22, for said synchronisation rule, must have a period Ttr=Tss (2k+1)/2; by choosing k=3 in this embodiment, Ttr=437.5 ms, which corresponds to a PT pattern stimulation frequency of 2.2 Hz, suitable for the transient stimulation. A trigger signal Tg 24 is used to synchronise the acquisition and the digitisation of the signal S which occurs at each rising edge of said trigger and for a time equal to the period of said trigger. The signal Tg, being synchronous with the frequency of reversal of the pattern PT, is generated by a same VPG module 15. Since two pattern reversals are present for each period Ttr, the trigger Tg has period equal to Ttr/2=218.75 ms. For each trigger the computer increases a sequential index n of acquisition so that the signal 25, S (n), represents the input signal 23 sampled and stored after the nth trigger.

It can be verified that if the periods Ttr and Tss comply the above mentioned synchronisation rule:

Ttr=Tss (2k+1)/2 with k being an integer, the sinusoidal second harmonic component F2Sss contained in the signal Sss has a 180° inverted phase for each successive acquisition, and the virtual stimulation transient signal Str does not vary. Based on this property, the sum of two successive acquisitions can be used to cancel the F2Sss component and to double the virtual signal Str. Similarly, the difference between two subsequent acquisitions can be used to cancel the signal Str and to double the F2Sss component. Therefore, the above mentioned synchronisation rule allows to obtain from pairs of successive acquisitions S (n) and S (n+1), a sinusoidal component 26 called F2Sss (n), and an approximate virtual transient stimulation signal 27, Stra (n), according to the following mathematical expressions:

F2Sss(n)=DFT2[S(n)−S(n+1)]/2; Stra(n)=NF4[S(n)+S(n+1))]/2

where n=1, 3, 5 . . . Nmax−1, is an odd integer, increasing with the number of acquired pairs; Nmax in this example has been set equal to 40; DFT2 is the Discrete Fourier Transform operator that returns the amplitude and the phase of the second harmonic component, in this example equal to 16 Hz.

While the component F2ss(n) is exactly reconstructed on the basis of the above mentioned expression, the signal Stra(n) can be affected by the interference of even harmonics higher than the F2, which are present in the steady-state Sss. In fact, the sum of subsequent acquisitions S(n)+S (n+1) cancels the sole second harmonic component F2 but does not cancel the higher even harmonics.

Therefore, the operator NF4 represents the IIR digital filter, that is notch filter type, with rejection band equal to +/−1 Hz, unit out-of-band gain, centred on the fourth harmonic F4 of the PS pattern frequency, in this example equal to 32 Hz. Any other harmonics which is over the fourth one are cancelled by means of the low-pass 30 Hz filter, implemented in the ADC device 13.

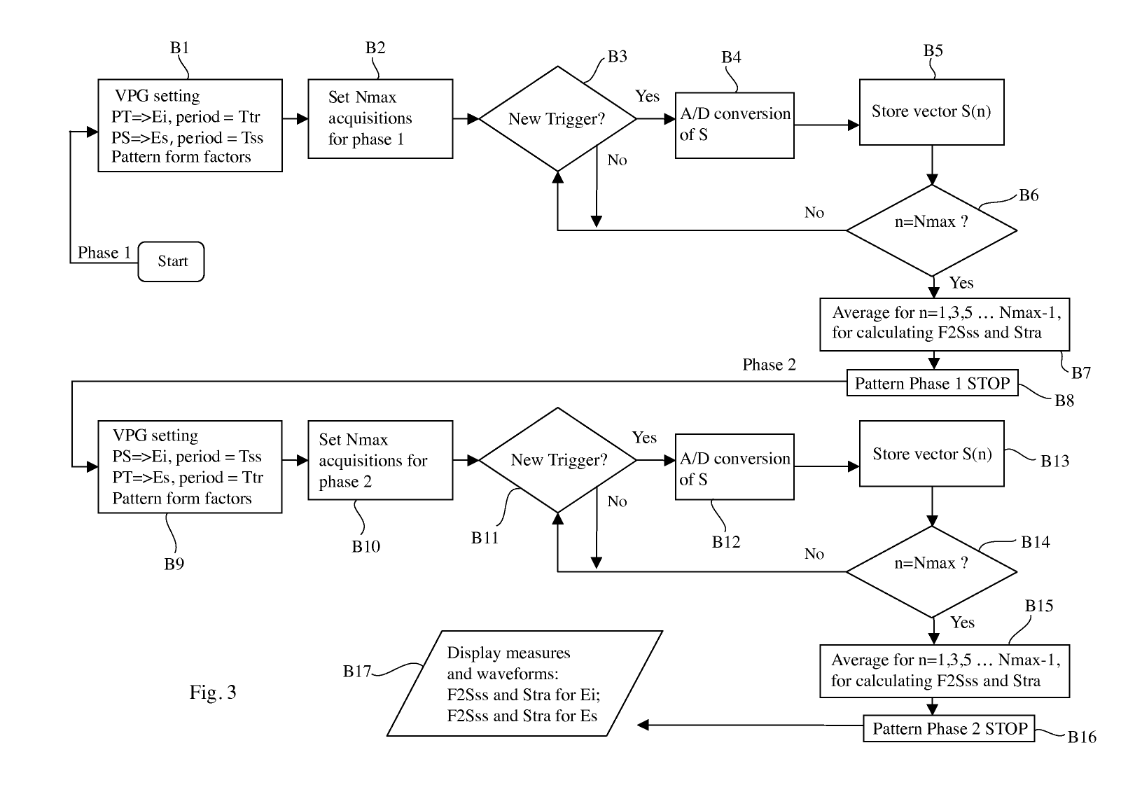

FIG. 3 shows the flow chart of the program executed by computer 14 in accordance with the above mentioned method, and is hereafter commented.

In phase 1 of the computer program, the program sets (block B1) in the generator VPG 15 the form factors of PT and PS patterns and the periods of stimulation of the same patterns, respectively Ttr=437.5 ms and Tss=125 ms, in order to trigger the transient stimulation of the lower retinal hemifield Ei made by PT, and the steady-state stimulation of the higher retinal hemifield Es made by PS.

Then a number of acquisitions Nmax is set in B2, able to provide an adequate signal-to-noise ratio. Typically this value is between 10 and 300 acquisitions. In the next block B3 the program waits for the trigger in order to acquire a new sampling vector, containing the signal S(n).

When the ADC device 13 completes the acquisition of said vector B4, the vector itself is stored in the RAM of the computer 14 (B5). A checking that the number Nmax of acquisitions has been reached (B6) follows. When the maximum number of acquisitions is reached, the phase 1 of the stimulation ends (B7), and the computer completes the processing of the acquired signals. The processing consists of obtaining the vectors F2Sss (n) and Stra (n) for each pair of acquisitions S (n) and S (n+1); the averages F2ss and Stra of these vectors are then calculated (B8) using the following formulas:

Stra=Σn Stra(n)/Nmax/2 for n=1,3, 5 . . . Nmax−1;

F2Sss=Σn F2Sss(n)/Nmax/2 for n=1, 3, 5 . . . Nmax−1;

When phase 1 ends, a phase 2 of the computer program is done in the same way as phase 1 in blocks B9 to B16, with a difference that the VPG generator 15 is programmed for repositioning the PT and PS stimulation patterns so that their positions on the display 17 are exchanged and, as a consequence, the stimulation hemifields Es and Ei are exchanged in their positions.

When phase 2 ends (B16), both transient and steady-state responses are available in B17 for each of two retinal hemifield Ei and Es subjected to stimulation. The whole visual angle stimulated is normally in a range between central 5 and 35 degrees.

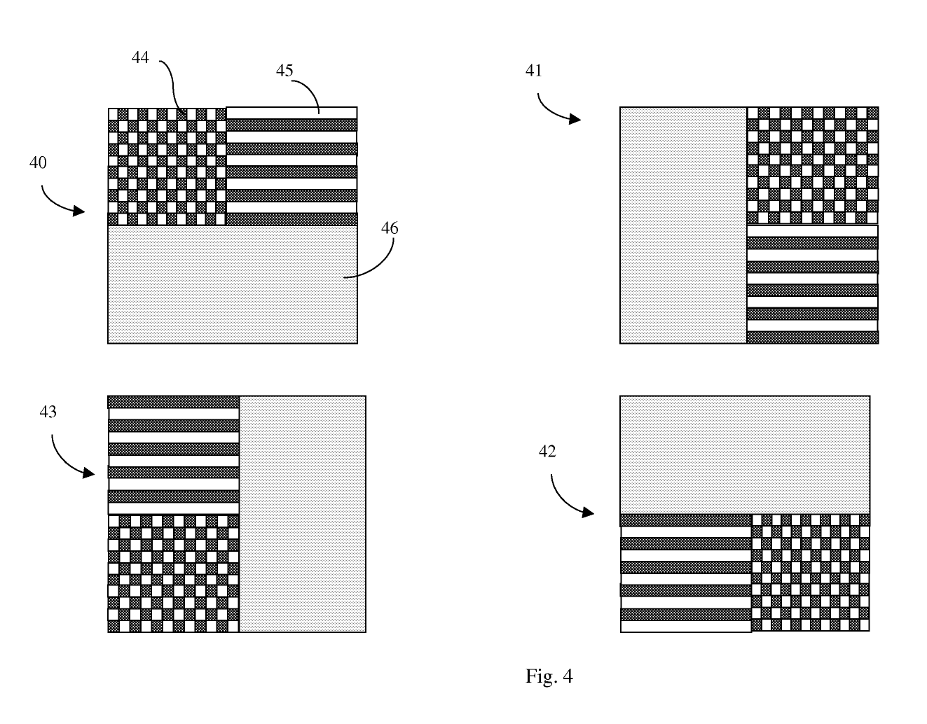

According to a suitable different embodiment using the visual stimulation depicted in FIG. 4, it is depicted how to extend the previously described method to the PERG and VEP stimulation and recording per four or more quadrants instead of two hemifields. In this representation patterns 44 and 45 stimulate two quadrants of the retina at a time, while the remaining surface 46 of the stimulation display is maintained at a constant luminance. The exam is carried out by means of a succession for simultaneous transient and steady-state stimulation according to the method described in FIG. 3, in accordance with a fixed or a predetermined repositioning pseudo-random sequence; by way of a non limiting example a sequence of patterns 40, 41, 42, 43 is depicted. When the examination is finished transient and steady-state responses of all said quadrants are available.

As the specificity of steady-state and transient PERG for the clinic diagnosis of glaucoma is scientifically well-known, the present invention allows to take advantage of a synergy of the reduced times of the simultaneous stimulation and of the double transient and steady-state mode in order to produce an improved apparatus for early glaucoma diagnosis. For this purpose, referring to the preferred embodiment in FIG. 1, an additional module of the program embedded in the acquisition computer 14 allows to calculate in accordance with the ISCEV standards and visualise the absolute value as well as their standard deviation with respect to a database of normal values of the following parameters:
Sss1/Sss2, the ratio between the amplitudes of steady-state response of the superior and inferior hemifields;
Stra1/Stra2, the ratio between the amplitudes of transient response of the superior and inferior hemifields;
Stra1/Sss1, the ratio between the amplitudes of transient and steady-state response of the same superior hemifield;
Stra2/Sss2, the ratio between the amplitudes of transient and steady-state response of the same inferior hemifield;
the amplitude of the whole steady-state response Sss1+Sss2 resulting from the sum of steady-state responses of the two hemifields;
the amplitude of the whole transient response Stra1+Stra2 resulting from the sum of transient responses of the two hemifields;
the phase delay of the steady-state response of the two hemifields;
the latency of the transient response of the two hemifields.

What is claimed is:

1. A method for visual stimulation and recording of the pattern electroretinogram (PERG) and of the visual evoked potentials (VEP), by means of transient and steady-state visual stimuli, said method comprising:
a) Generation and simultaneous projection, by means of a visualization means, of two distinct and separate patterns, one of which is a transient pattern and the other one is a steady-state pattern, both having average luminance constant over time, wherein each of said patterns produces a stimulation of a zone of the retina of a subject.
b) Modulating or inverting the contrast of each one of the said pattern, wherein the transient pattern has period Ttr and the steady-state pattern has period Tss, according to the following synchronization rule:
Ttr=Tss(2k+1)/2 where k is a predetermined integer.
c) Acquiring the signals from the visual electrophysiological response of the subject submitted to said two patterns stimulation.
d) Analyzing the acquired signals, by means of an algorithm capable to reconstruct the second harmonic component of the steady-state signal and the transient signal of said zones.
e) Repositioning of said patterns on the visualization means and repeating steps a, b, c, d in order to stimulate and record the response of a plurality of retinal zones.

2. An apparatus for visual stimulation and recording of the pattern electroretinogram (PERG) and of the visual evoked potentials (VEP), by means of transient and steady-state visual stimuli, consisting of a visualization means able to simultaneously project on the retina two distinct and separate patterns, one of which is a transient pattern and the other one is a steady state pattern, both having average luminance constant over time, wherein each one of said patterns produces a stimulation of one zone of the retina of a subject, an acquisition means of the signals from the visual electrophysiological response of the subject submitted to said two pattern stimulation, said means also comprising a plurality of electrodes applied to the subject, a preamplifier, a filter and an analog to digital conversion device, a data acquisition and control computer, and wherein said apparatus comprises:
a) a pattern generator that provides for the contrast inversion of each one of the said patterns, where the transient pattern has period Ttr and the steady-state pattern has period Tss, according to the following synchronization rule:
Ttr=Tss(2k+1)/2, with k predetermined integer.
b) a software algorithm able to reconstruct the second harmonic component of the steady-state signal and the transient signal for said zones.
c) A program that executes the repositioning of said patterns on the visualization means and the repetition of steps a,b,c in order to stimulate and record the response of a plurality of retinal zones.

3. Apparatus according to claim 2 wherein said patterns are repositioned on the visual stimulation means, after a packet of acquisitions ranging between a minimum of 10 and a maximum of 300 acquisitions.

4. Apparatus according to claim 2 wherein the value of the integer constant k is comprised between 1 and about 80.

5. Apparatus according to claim 2 wherein said patterns fill up the upper and lower half respectively of a stimulation means, in order to stimulate simultaneously two hemifields of the retina.

6. Apparatus according to claim 2 wherein the retina is divided into four quadrants and the transient and steady-state pattern stimuli simultaneously stimulate, according to a predetermined sequence, two quadrants at time of the retina, whereas the rest of the stimulator surface is kept at a constant luminance.

7. Apparatus according to claim 2 wherein, for the purpose of the early glaucoma diagnosis, a program is present that, according to the ISCEV standards, calculates and displays, both in terms of absolute value and in terms of standard deviation with respect to a database of normal values, the following parameters:
Sss1/Sss2, the ratio between the amplitudes of steady-state response of the superior and inferior hemifields;
Stra1/Stra2, the ratio between the amplitudes of transient response of the superior and inferior hemifields;
Stra1/Sss1, the ratio between the amplitudes of transient and steady-state response of the same superior hemifield;
Stra2/Sss2, the ratio between the amplitudes of transient and steady-state response of the same inferior hemifield;
the amplitude of the whole steady-state response Sss1+Sss2 resulting from the sum of steady-state responses of the two hemifields;
the amplitude of the whole transient response Stra1+Stra2 resulting from the sum of transient responses of the two hemifields;
the phase delay of the steady-state response of the two hemifields;
the latency of the transient response of the two hemifields.
